(19) United States
(12) Patent Application Publication
(10) Pub. No.: US 2015/0342685 A1
(43) Pub. Date: Dec. 3, 2015

(54) SURGICAL DRAPE WITH STERILE SYSTEM ACCESS

(71) Applicant: AVENT, INC., Alpharetta, GA (US)

(72) Inventor: Richard I. Livesey, Wheatley (GB)

(21) Appl. No.: 14/287,563

(22) Filed: May 27, 2014

(51) Int. Cl. A61B 19/08

(52) U.S. Cl. CPC A61B 19/081; A61B 19/088; A61B 2019/082

(57) ABSTRACT

A surgical drape for use during surgery on a patient includes a transparent portion that used to cover an instrument array. The drape also has a hole containing panel with a flap whereby at least one line from the instrument array can pass through the drape to the patient. The flap folds down and covers the hole through which the line passes to help maintain the sterile field.

Various configurations of disposable surgical drapes are known in the art for keeping a surgical site on a patient sterile during a surgical procedure. A reinforcement area is often placed around a fenestration or an edge of disposable surgical drapes to provide structural strength and to absorb bodily fluids from the surgical site. Many disposable drapes also include a number of layers of different materials for the drape area and reinforcement area, with each layer providing a different property to the drape. For example, spunbond fabrics, meltblown fabrics, and polymer films have been used as layers in disposable drapes.

Certain surgical procedures involve the injection of contrast fluids. Past practice has been to inject the fluid with a hand held syringe into a line introduced into the femoral or radial artery. Contrast fluid is very viscous so relatively high pressure must be used to infuse the fluid and this requires good hand strength on the part of the physician. Contrast fluid is also expensive and any unused portion of the fluid must be disposed of since it will have entered the patient field during surgery. A relatively new device is becoming more widely used and allows for the saving of the contrast fluid not used on the patient. This device, known as the ACIST CVi contrast delivery system, injects the contrast fluid via a high pressure line from the ACIST system and is controlled by a simple hand control piece, therefore making it less strenuous for the physician to infuse. In addition, any remaining contrast media can be used for another patient as it is stored in a small glass bottle on the ACIST system, outside of the patient field. The ACIST system has a touch screen display (CRT) and lines to deliver the fluid to the patient and a line to allow for the hand control of the fluid flow.

The problem that has arisen is the connection between the system and the patient, since the line(s) between them must pass from inside the patient field to outside the patient field. One practice that has arisen to address this problem is to poke holes through the drape with a pen or a sharp instrument and run the controller line and the high pressure line through the holes, with the CRT remaining uncovered and outside the sterile field. Another solution has been to run the line above the drape with no specific barrier between the sterile field and the non-sterile field, hanging it from the operating room ceiling or a piece of equipment. Neither solution is ideal and there is no standard draping system for this system and procedure.

As such, a need currently exists for a surgical drape that has access provided for use with an ACIST CVi or similar system that is easy to use and provides separation between the patient and the equipment.

### SUMMARY

Various features and advantages of the disclosure will be set forth in part in the following description, or may be obvious from the description, or may be learned from practice of the disclosure.

In accordance with aspects of the disclosure, a surgical drape is provided for use during surgery of a patient. The drape includes a material sheet having a size and configuration for covering at least a portion of the patient as well as an instrument array, during the procedure. The drape includes pre-formed holes allowing the lines from the instrumentation array to the patient to pass through the drape. The drape also includes a flap that covers the holes after the lines have been passed through the holes, in order to help maintain the sterile field around the patient.

### BRIEF DESCRIPTION OF THE DRAWINGS

FIG. 1 is a view of a surgical drape covering a patient and an instrument array, showing the lines passing through the flap in the drape.

FIG. 2 is a plan view of an embodiment of the drape showing the location of various features.

FIG. 3 is a plan view of the holes and flap on the drape.

FIG. 4 is a plan view of an alternative embodiment of the drape showing the location of various features.

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. Thus, it is intended that the present disclosure cover such modifications and variations as come within the scope of the appended claims and their equivalents.

Surgical drapes formed in accordance with the present disclosure can generally possess any of a variety of sizes and shapes, depending on the particular use of the drape and on its desired properties. For example, certain surgical drape configurations are described in U.S. Pat. No. 6,055,987 to Griesbach, et al., which is incorporated herein in its entirety by reference thereto for all purposes.

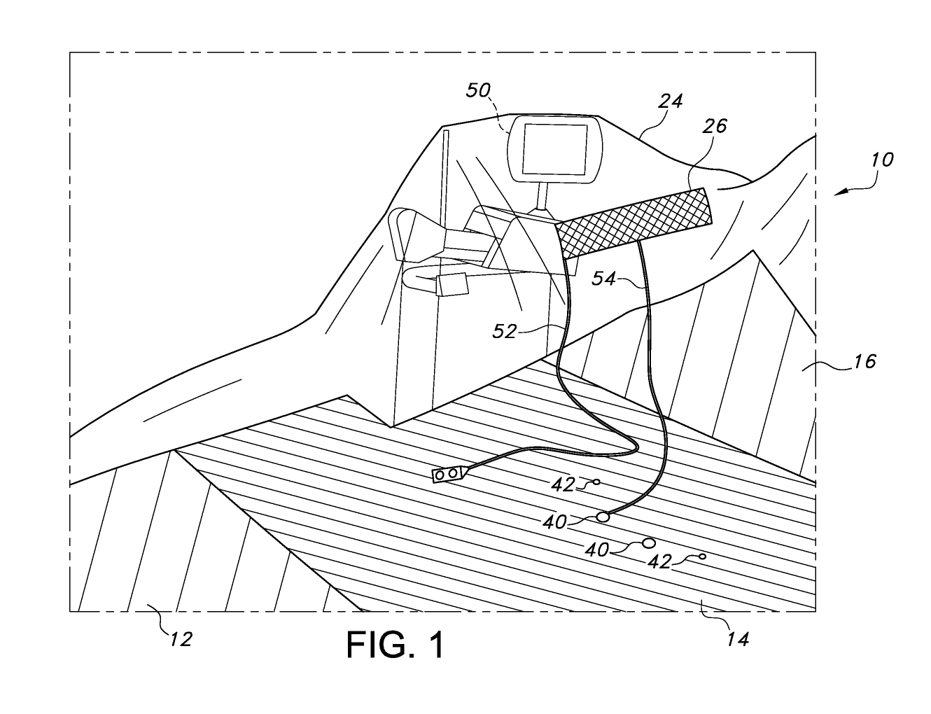

Various embodiments of surgical drapes incorporating aspects of the disclosure are depicted in the figures as drapes 10 for covering a patient (FIG. 1) during a surgical procedure. The drapes 10 may be formed of any material or combination of materials defining a drape sheet material commonly used in the art for disposable surgical drapes, garments, covers, and so forth.

In general, the drape sheet may be made from a wide variety of materials, including, for example, woven, reusable fabrics and nonwoven disposable fabrics or webs. Nonwoven materials suitable for use with the present disclosure include, for example, multilayer laminates such as a spunbond/meltblown/spunbond (“SMS”) material. An example of a suitable fabric is disclosed in U.S. Pat. No. 4,041,203.

As used herein the term “nonwoven fabric or web” means a web having a structure of individual fibers or threads that are randomly interlaid, but not in an identifiable manner or pattern as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91).

As used herein the term “spunbond fibers” or “spunbonded fibers” refers to small diameter fibers which are formed by extruding molten thermoplastic material as filaments from a plurality of fine, usually circular capillaries of a spinneret with the diameter of the extruded filaments then being rapidly reduced, for example, as in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters larger than 7 microns, more particularly, between about 10 and 20 microns.

As used herein the term “meltblown fibers” means fibers formed by extruding a molten thermoplastic material through a plurality of fine, usually circular, die capillaries as molten threads or filaments into converging high velocity, usually hot, gas (e.g. air) streams that attenuate the filaments of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin et al. Meltblown fibers are microfibers that may be continuous or discontinuous, are generally smaller than 10 microns in average diameter, and are generally tacky when deposited onto a collecting surface.

As used herein “multilayer laminate” means a laminate wherein some of the layers are spunbond and some meltblown such as a punbond/meltblown/spunbond (SMS) laminate and others as disclosed in U.S. Pat. No. 4,041,203 to Brock et al., U.S. Pat. No. 5,169,706 to Collier, et al, U.S. Pat. No. 5,145,727 to Potts et al., U.S. Pat. No. 5,178,931 to Perkins et al. and U.S. Pat. No. 5,188,885 to Timmons et al. Such a laminate may be made by sequentially depositing onto a moving forming belt first a spunbond fabric layer, then a meltblown fabric layer and last another spunbond layer and then bonding the laminate in a manner described below. Alternatively, the fabric layers may be made individually, collected in rolls, and combined in a separate bonding step. Such fabrics usually have a basis weight of from about 0.1 to 12 osy (6 to 400 gsm), or more particularly from about 0.75 to about 3 osy. Multilayer laminates may also have various numbers of meltblown layers or multiple spunbond layers in many different configurations and may include other materials like films or coform materials, e.g. SMMS, SM, SFS, etc.

As used herein, the term “coform” means a process in which at least one meltblown diehead is arranged near a chute through which other materials are added to the web while it is forming. Such other materials may be pulp, superabsorbent particles, cellulose or staple fibers, for example. Coform processes are shown in commonly assigned U.S. Pat. No. 4,818,464 to Lau and U.S. Pat. No. 4,100,324 to Anderson et al. Webs produced by the coform process are generally referred to as coform materials.

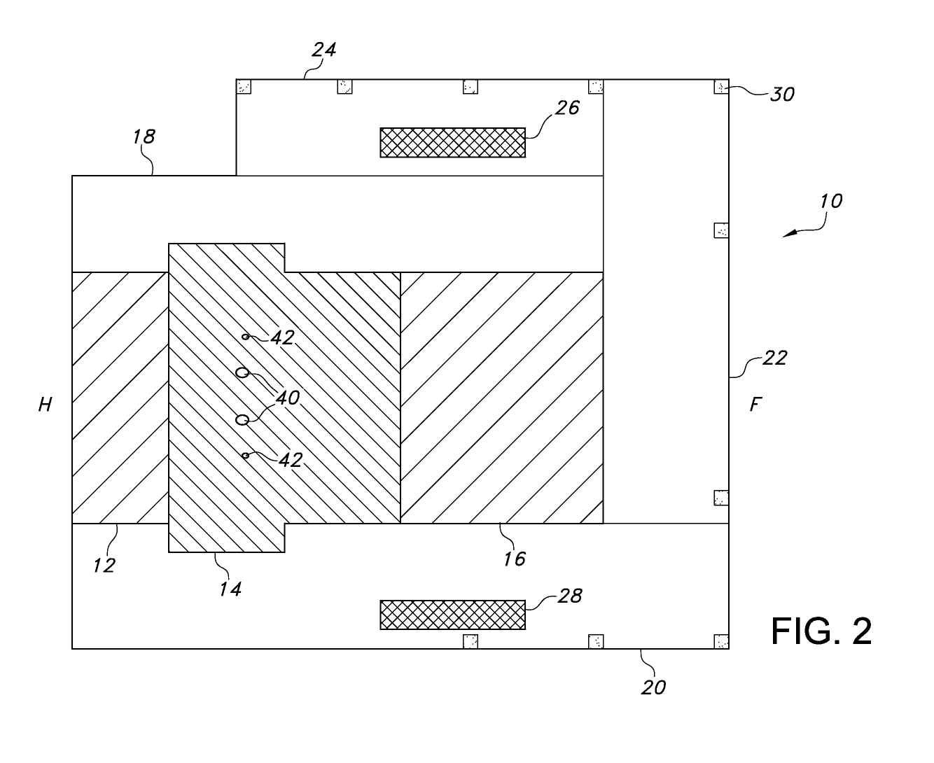

In one embodiment, the drape 10 includes femoral fenestration openings 40 and radial fenestrations 42 that can be placed over an operating site during surgery as is well known in the art. The fenestrations 40, 42 have a size, shape, and location that varies as a function of the particular type of surgical procedure the drape 10 is intended for. For example, drapes intended for use in femoral angiography procedures may include one or two generally circular fenestrations 40, about 12 or 13 mm in diameter, as indicated in FIG. 2. The radial fenestrations 42 are generally oval shaped and 12 mm by 7 to 8 mm in diameter.

Instrument arrays may be used to monitor the progress of surgery or to administer fluids, like angiography contrast fluid, to a patient. The instrument array may contain, for example, a tough screen display (CRT) and other associated instrumentation to deliver the contrast fluid. A relatively new device is being used in angiography and involves the use of such an instrument array. This device, known as the ACIST CVi contrast delivery system, injects the contrast fluid via a high pressure line from the ACIST system and is controlled by a simple hand control piece, therefore making it less strenuous on the user to infuse. In addition, any remaining contrast media can be used for another patient as it is stored in a small glass bottle on the ACIST system, outside of the patient field. The ACIST system has a touch screen CRT and lines to deliver the fluid to the patient and a line to allow for the hand control of the fluid flow.

The drape 10 provided herein should include viewing panels (e.g., items 18, 20, 22, 24 in FIG. 2) made of a transparent, fluid resistant material, such as polyethylene film, to cover the patient and the instrument array 50 and for easy viewing of the instrument array 50 through the transparent film as illustrated in FIG. 1. Commonly used transparent film is generally from 10 to 60 microns in thickness, more particularly about 40 microns in thickness.

The surgical drape 10 of the present disclosure may include reinforcement panels 12, 14, 16 superimposed on and affixed in any suitable and appropriate manner to a base sheet, or bonded to the edges of the other panels (e.g. 18, 20, 22, 24). The width and length and materials of construction of the reinforcement panels may vary depending on the intended use of the drape 10. The reinforcement panels may be formed from a variety of materials, such as nonwoven fabrics, multilayer laminates (e.g. SMS), fluid-absorbing materials and combinations thereof. The reinforcement panels may be backed by a fluid-repellent or fluid-impervious film layer. The film-layer side or lower surface of the reinforcement panels may be secured to the upper surface of a base sheet by any conventional means, including adhesive, stitching, thermal or ultrasonic bonding techniques. The upper surface of the reinforcement panels remains exposed and available to absorb fluids emitted from the surgical site. The fluid-impervious film layer prevents the passage of blood and other body fluids through the reinforcement panels and a base sheet, if present.

Strips or squares of adhesive 30 may be positioned around the periphery of the drape 10 to adhere the drape to the patient or to the instrument array 50. The tacky and pressure-sensitive adhesives used may be of any biologically acceptable adhesive. Examples of such adhesive materials are described in U.S. Pat. No. 3,669,106 entitled “Surgical Drape with Adhesive Attachment Means” to Schrading et al.

Some or all of the materials used to form the drape may be constructed so as to be hydrophilic or hydrophobic, and may be chemically treated to achieve the desired water absorbency properties. For instance, one or more materials may be treated with a surfactant in a manner such as described in U.S. Pat. No. 5,540,979.

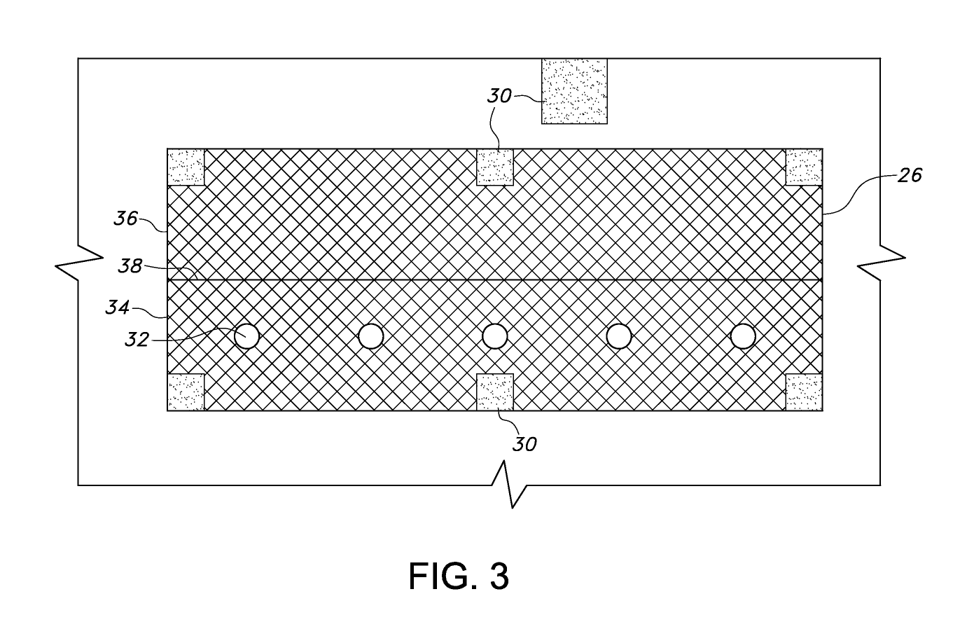

Referring to the figures, there is visible an instrument array 50 below a viewing panel 24 section of the drape 10 in FIG. 1. The array 50 has two lines 52, 54 that are run through the pre-formed holes 32 in the first hole panel 26 of the drape 10. (Note that the first hole panel 26 and a small area of surrounding drape 10 are shown enlarged in FIG. 3.) One of the lines is the controller line 52 and the other is the high pressure fluid line 54 that continues to the patient through one of the fenestrations 40. The first hole panel 26 has a flap 36 and a hole containing section 34. The flap 36 is only connected to the drape 10 at the fold 38. In use, the flap 36 is lifted and the lines 52, 54 inserted through any of the holes 32 and run to the array 50. The flap 36 folds down over the hole containing section 34 of the first hole panel 26 to shield the lines 52, 54 and the holes 32 and so maintain the sterile field of the patient after the lines have been inserted through the holes 32. The hole panel(s) may be made of the same materials as the reinforcement panel, as discussed above.

The first hole panel 26 is shown in FIG. 2 as located on the upper side of the drape 10 since this is the most common location of the array 50 for surgery. Individual physician preference or other factors may dictate the array 50 be placed at another location. For this reason a second hole panel 28 is also provided on the other side of the drape 10. Since the second hole panel 28 is on the opposite side of the patient, it is rotated 180 degrees so that the flap 36 folds down towards the patient. The two hole panels 26, 28 need not be the same size nor contain the same number of holes 32. In some embodiments the first hole panel 26 has five holes 32 and the second hole panel 28 has three holes 32, for example. The hole containing sections in this view are shown with the flaps closed so the holes are not visible.

In one embodiment (FIG. 2), the drape has an overall length of 340 cm in its longest dimension. At its head end (H in FIG. 2) it is 260 cm wide. The foot end (F in FIG. 2) width is 295 cm. The panels 12, 14 and 16 have a combined length of 275 cm with panel 14 being slightly wider by 15 cm on each side than panels 12 and 16. The panels 12, 14, 16 may desirably be made of an SMS fabric with an absorbent layer added to panel 14 to aid in controlling blood and other fluids. The viewing panel 24 has a width of 50 cm and ends 150 cm short of the head end.

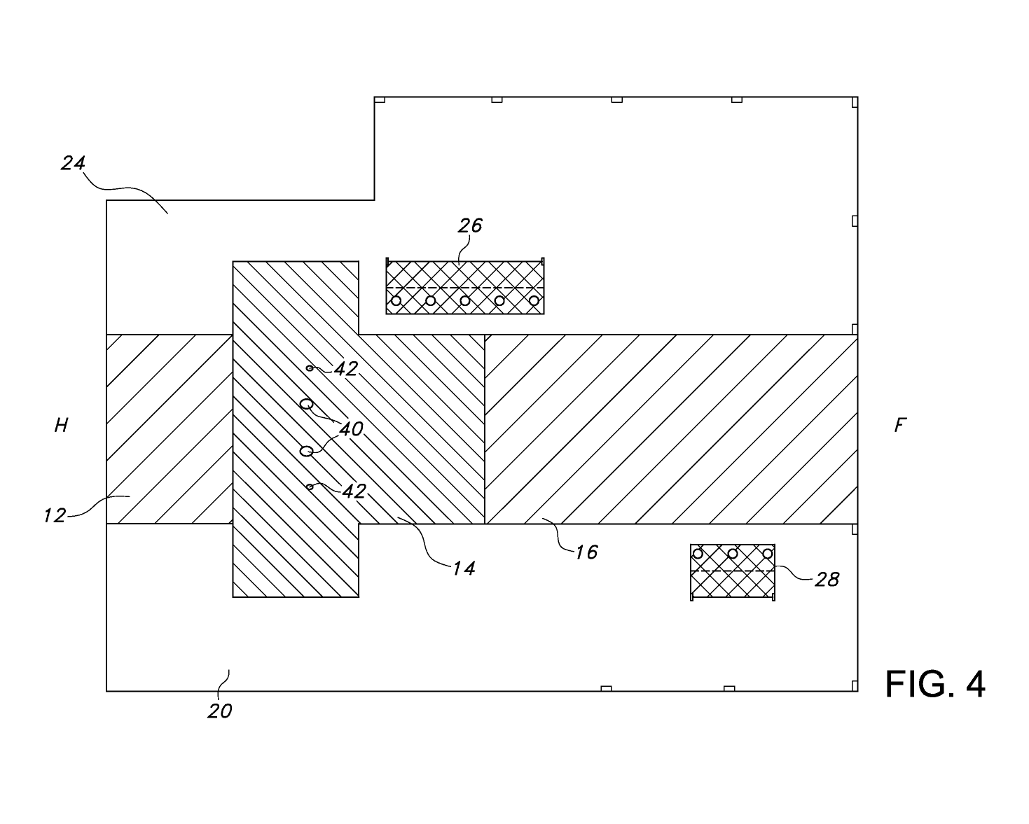

In another embodiment (FIG. 4), the drape has an overall length of 363 cm in its longest dimension. At its head end (H in FIG. 4) it is 238 cm wide. The foot end (F in FIG. 4) width is 288 cm. The panels 12, 14 and 16 extend for the entire length of the drape (363 cm) so that there is no continuous transparent portion along the head or foot of the drape. Part of panel 14 is slightly wider by 35.5 cm on each side than panels 12 and 16, which are both 91 cm wide. The widened area of panel 14 extends for 61 cm along the length. The panels 12, 14, 16 may desirably be made of an SMS fabric with an absorbent layer added to panel 14 to aid in controlling blood and other fluids. There is a transparent viewing panel 24 that is 363 cm long and that has a width of 115 cm at the foot end and 65 cm at the head end, reducing in width 130 cm short of the head end. The alternative transparent viewing panel 20 is 81 cm wide and 363 cm long. The first hole panel 26 hole containing section 34 is 76 cm long and 12.5 cm wide and the flap 36 is the same size. The second hole panel 28 hole containing section is 41 cm long and 12.5 cm wide and the flap is the same size. The hole containing sections in this view are shown with the flaps open so the holes are visible.

The disclosed drape enables the user to pass lines from the sterile to non-sterile fields through the drape. It also allows a sterile user (i.e. nurse) the option to set up the system without the assistance of a non-sterile nurse and allows the user to cover and use the touchscreen from the ACIST CVi system using only the patient drape and without the need for additional screen covers.

It should be appreciated by those skilled in the art that various modifications and variations can be made to the embodiments illustrated and described herein without departing from the scope and spirit of the disclosure. It is intended that the disclosure include such modifications and variations as come within the scope of the appended claims and their equivalents.

What is claimed is:

1. A drape for use during surgery on a patient, comprising:
a drape configured for covering at least a portion of the patient during surgery as well as for covering an instrument array, the drape portion covering the instrument array being transparent; and
at least one hole panel comprising a hole containing portion and a flap, wherein a line for the delivery of a fluid to the patient can pass from the instrument array through a hole in the hole containing portion to the patient, and the flap covers the hole containing portion.

2. The drape of claim 1 wherein said instrument array is a contrast delivery system for angiography.

3. The drape of claim 1 wherein said drape material is liquid impermeable.

4. The drape of claim 1 further comprising a second hole containing portion in an alternative location of said drape.

5. The drape of claim 1 wherein said drape has at least one reinforcement panel that is made from a material selected from the group consisting of nonwoven fabrics, multilayer laminates, fluid-absorbing materials and combinations thereof.

6. The drape of claim 5 wherein the at least one reinforcement panel is backed by a fluid-repellent or fluid-impervious film layer.

7. The drape of claim 1 wherein said hole containing portion is attached to a transparent portion of said drape.

8. The drape of claim 1 wherein said hole panel is made from a material selected from the group consisting of nonwoven fabrics, multilayer laminates, fluid-absorbing materials and combinations thereof.

9. A drape for use during an angiography procedure comprising:
a drape configured for covering at least a portion of a patient as well as for covering a contrast delivery system, the drape portion covering the contrast delivery system being transparent; and
at least one hole panel comprising a hole containing portion and a flap, wherein a fluid delivery line and a controller line can pass from the contrast delivery system through holes in the hole containing portion, and the flap folds down over the lines where they pass through the hole containing portion.

10. The drape of claim 9 wherein said contrast delivery system has a CRT that remains outside of the sterile field.
